(19) United States
(12) Patent Application Publication
(10) Pub. No.: US 2004/0162320 A1
(43) Pub. Date: Aug. 19, 2004

(54) Solid composition containing nisoldipine a mixture of polyethylene oxides and an antioxidant

(76) Inventor: Pawan Seth, Irvine, CA (US)

Correspondence Address: HARNESS, DICKEY & PIERCE, P.L.C., P.O. BOX 828, BLOOMFIELD HILLS, MI, 48303, US

(21) Appl. No.: 10/367,655

(22) Filed: Feb. 14, 2003

(51) Int. Cl. A61K 31/455; A61K 31/08

(52) U.S. Cl. 514/355; 514/723

(57) ABSTRACT

The invention provides a solid composition of nisoldipine and a mixture of two polyethylene oxides having specific viscosities. The invention also provides a process for manufacturing said composition.

### BACKGROUND OF THE INVENTION

The present invention relates to novel solid compositions, notably pharmaceutical compositions, containing a mixture of polyethylene oxide (PEO), an antioxidant and Nisoldipine as active ingredient, and to methods for their preparation.

Certain medicaments need to be formulated in so-called delayed-release or sustained release form. Polyethylene oxide referred to as PEO below is moreover known as a component of medicaments in tablet form designed to be administered by oral route. This compound is marketed by the DOW Corporation under the commercial name Polyox®. The use of PEO for formulating medicaments has furthermore been the subject matter of many earlier patents.

The abstract of the U.S. Pat. Nos. 4,404,183 and 4,343,789 discloses two embodiments of a sustained release composition. In the first embodiment, the composition contains PEO, the active ingredient in an amorphous form, and a basic component. In the second embodiment, the active ingredient is nicardipine in an amorphous state, it being possible to omit the basic component.

There is a need for providing various nisoldipine compositions with dissolution profiles which can be adjusted in a range from slow to fast release according to the effect targeted for the clinical trials. None of the above documents teaches or suggests the instant invention.

### SUMMARY OF THE INVENTION

Applicant has found that nisoldipine compositions containing PEO having different dissolution profiles could be obtained by mixing two PEO, each one having different viscosity, the composition further comprising an antioxidant, such as vitamin. The present invention thus provides a solid composition comprising, by weight based on the total weight of the composition:

(a) from 5 to 45% of nisoldipine;

(b) from 50 to 90% of polyethylene oxide; said polyethylene oxide being a mixture comprising at least two polyethylene oxides with different viscosity range, at least one of a low viscosity in a range between 400 and 2,000 cp, and at least another of a high viscosity in a range between 4,000 and 20,000 cp;

(c) the balance consisting of conventional additives.

### BRIEF DESCRIPTION OF THE DRAWINGS

FIG. 1 is a graphical illustration of dissolution profiles related to formulations A, B, with one Polyox® of different viscosity, and Sular®.

FIG. 2 is a graphical illustration of different dissolution profiles related to examples formulations C, D, E, containing different proportion of a mixture of two Polyox® having different viscosity, and Sular®.

### DETAILED DESCRIPTION

The invention will apply to solid compositions of nisoldipine. In the framework of this invention, the expression “solid composition” means that the composition is in a granule, a tablet or mini-tablet form, these in their turn being able to be encapsulated using for example the conventional hard gelatin. The content of Nisoldipine in the composition is from 5 to 45% by weight based on the total weight of the composition, preferably 5 to 25%.

According to the invention, the active ingredient nisoldipine is preferably not in amorphous form; it is generally in crystalline form. The expression “not in amorphous form” should be understood in its conventional meaning. Various sources give a definition of this term “amorphous” as meaning non-crystalline, lacking the lattice structure characterizing the crystalline state. The following references, which provide a definition of the term amorphous (or the opposite thereof) are, in a on-limiting manner: Hawley's, Condensed Chemical Dictionary, 12th Edition, p. 71; Handbook of Chemistry and Physics, 65^{th} Edition, F-67; The Theory and Practice of Industrial Pharmacy, 1970, pp. 253-255; Remington's Pharmaceutical Sciences, 14th Edition, p. 182; General Chemistry 1992, pp.314-315; Encyclopedia of Pharmaceutical Technology, vol 1, pp.12-13.

The particles size can vary within broad limits, up to e.g. 50 microns. In a preferred embodiment the nisoldipine is in a micronized form with a particle average size of 1 to 10, typically about 3, microns.

In the composition according to the invention, the mixture of PEO comprises two PEOs each one with different viscosity; the first one is a high viscosity polyethylene oxide while the second one is a low viscosity polyethylene oxide. The high viscosity can be comprised between 4,000 and 20,000, preferably 8,800 and 17,600 cp. One preferred high viscosity PEO has an approximate molecular weight of about 900,000. The low viscosity can be comprised between 400 and 2,000, preferably between 600 and 1,200 cp. One preferred low viscosity PEO has approximate molecular weight of about 300,000. The method to determine the viscosity of PEO batches is provided by the supplier (Dow Chemical). The viscosity specifications were achieved with a Brookfield RVF viscosimeter for a 5% aqueous solution. The amount of PEOs may vary from 50 to 90%, preferably from 60 to 75%, by weight based on the total weight of the composition.

According to one embodiment, the composition further contains a non-volatile antioxidant, esp. those which do not sublimate during the drying of the granules in the granulation step of the final formulation. One example is Vitamin E. The amount may be from 0.01 to 2% by weight based on the total weight of the composition. In the composition according to the invention the balance consisting of conventional additives comprises classical excipients, such as microcrystalline cellulose, lactose, ascorbic acid, pigments, plastifying agents, magnesium stearate, glycerol behenate, sodium stearyl fumarate, lubricants, fillers, extenders, desintegrating agents, binders, water-soluble polymers (e.g. PVP) and so on. Obviously, other conventional additives known to those skilled in the art can be employed. The weight % are given with respect to the final composition, excluding any cosmetic coating on it.

In order to obtain the desired profile according to the clinical trial purpose, Applicant has found that it is possible to obtain various dissolution profiles for nisoldipine compositions by adjusting and varying dependently the proportion of each Polyox® within the same total amount of Polyox®. According to one embodiment, the weight ratio low viscosity polyethylene oxide to high viscosity polyethylene oxide from 9:1 to 1:9, preferably from 8:2 to 2:8. A most preferred ratio is about 1:4.

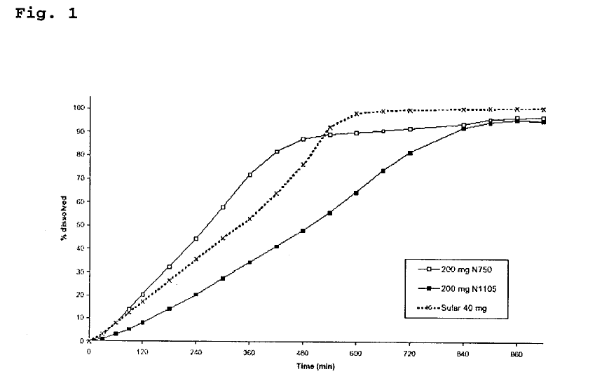

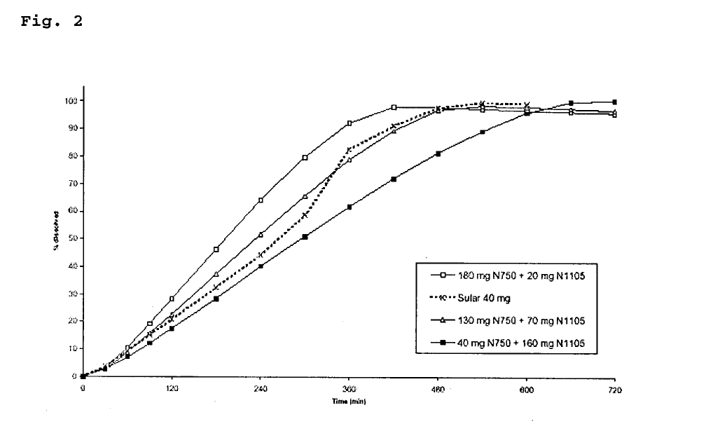

In the two figures, Sular® 40 mg is the reference product manufactured by Zeneca and presents a dissolution profile which could be targeted for clinical trials. According to FIG. 1, it can be observed that the profile determined by the Sular® formulation is situated between the two formulations profiles A and B. Formulation A which contains the high viscosity PEO named Polyox® N1105 shows the slower dissolution profile and formulation B which contains the low viscosity PEO named Polyox® N 750 shows the faster dissolution profile. According to FIG. 2, it can be observed that the profile can be fitted by adjusting the mixture of two PEOs. In FIG. 2: the formulation with 180 mg Polyox® N750+20 mg Polyox® N1105 is formulation C described in the examples; the formulation with 130 mg Polyox® N750+70 mg Polyox® N1105 is formulation D described in the examples and the formulation with 40 mg Polyox® N750+160 mg Polyox® N1105 is formulation E described in the examples.

Applicant has found that the target dissolution profile for nisoldipine compositions according to the invention is related to the value of the blend parameter P which can be obtained by the following equation:

P=a.Log(x)+b.Log(y)

with

a: amount of high viscosity polyethylene oxide in mg;

b: amount of low viscosity polyethylene oxide in mg;

x: viscosity of high viscosity polyethylene oxide in cp;

y: viscosity of low viscosity polyethylene oxide in cp;

Therefore in a preferred embodiment the compositions of the invention are those where P is comprised from 700 to 800, preferably 750 to 780, for a 200 mg polyethylene oxide amount.

The present composition can be obtained by any conventional method known to those skilled in the art such as, for example direct compression after mere mixing the dry ingredients, moist or wet granulation involving the use of a granulation liquid, and dry granulation involving a densification phase for the dry mixture. A method for preparing a solid composition according to the invention comprises the steps of:

(i) mixing in the dry state and for a sufficient time, the active ingredient, the mixture of the two polyethylene oxide and optionally, one or several additives;

(ii) adding a solution, e.g. alcoholic solution, with optionally one or several additives, followed by mixing for a sufficient period of time;

(iii) granulating the mixture obtained from step (ii), e.g. by passage through a suitable sieve;

(iv) drying the granules thus formed for a sufficient period of time.

The alcohol employed in the alcoholic solution is preferably isopropanol. The solvent is eliminated by drying at one point or another in the process, and is substantially not encountered in the final composition. The choice of mixing times, apparatus used, sieve mesh, and other operating conditions are within the province of the normal knowledge of those skilled in the art. The granules thus obtained can then be compressed into tablets by methods known in the art, with the aid of classical processing aids if need be.

### EXAMPLES

The following examples illustrate the invention without limiting it.

The following compositions A to E are prepared with different amounts of Polyox®. Formulation A contains only one PEO with high viscosity: Polyox® N1105: 12340 cp at 25° C. with spindle at 2 rpm. Formulation B contains only one PEO with low viscosity: Polyox® N750: 720 cp at 25° C. with spindle at 10 rpm. Formulations C, D, E, according to the invention contain different quantities of each two Polyox® described above within the same total amount of PEO.

Preparation of the Granules

The granules have the following formulations.

| Compounds | A mg | B mg | C mg | D mg | E mg |
|---|---|---|---|---|---|
| Nisoldipine | 40 | 40 | 40 | 40 | 40 |
| Polyox N1105 | 200 | — | 20.00 | 70.00 | 160.00 |
| Polyox N750 | — | 200 | 180.00 | 130.00 | 40.00 |
| Avicel PH 101 | 45.00 | 45.00 | 45.00 | 45.00 | 45.00 |
| Plasdone K29-32 | 10.50 | 9.90 | 9.90 | 9.90 | 9.90 |
| Isopropanol | 80 | 80 | 80 | 80 | 80 |
| Vitamin E | — | 0.60 | 0.60 | 0.60 | 0.60 |
| Total weight | 295.5 | 295.5 | 295.5 | 295.5 | 295.5 |
| P parameter | 818 | 572 | 597 | 658 | 769 |

The nisoldipine, the two PEO and microcrystalline cellulose (Avicel PH101) are weighted and added to a kneader. Mixing in a dry state is carried out for 5 mn. Plasdone and vitamin E are dissolved in isopropanol. Then this solution is added to the mixture and followed by mixing by 5 minutes. Granulation is achieved by passing through a sieve of 1.6 mm mesh. The granules are then dried in an oven at 45° C. during 5 hours.

Preparation of the Tablets

The following composition is prepared:

| Ingredients | amount per dosage form (mg): |
|---|---|
| granules | 295.5 mg |
| colloidal silicon dioxide | 1.50 mg |
| Sodium stearyl fumarate | 3.00 mg |
| Total | 300.0 mg |

After weighing, the granules, Sodium stearyl fumarate and colloidal silicon dioxide are passed through a sieve of 0.8 mm mesh. Then mixing in the dry state is performed during 5 minutes. Tablets are obtained by compression using a Fette P2 machine.

Coating of the Tablets

The above tablets can be further coated by a classical Opadry coating according to the following formulation.

| Ingredients | amount per dosage form (mg): |
|---|---|
| Tablets cores | 300.0 mg |
| Opadry II | 7.30 mg |
| PEG 1450 | 0.70 mg |
| Purified water | 56.00 |
| Total dry weight | 308.0 mg |

Polyethylene glycol is dissolved in purified water. Opadry II is added to the solution and the solution is kept under stirring during 45 min. After sieving through a 350 micrometer sieve, the solution is sprayed onto the tablets in a coating pan Vector LCDS with the following spraying parameters.

| | |
|---|---|
| Nozzle: | 1.2 mm |
| Distance Bed-Nozzle | 7.5 cm |
| Inlet Temperature | 52° C. |
| Outlet Temperature | 40° C. |
| Spraying Rate | 6 g/min |
| Atomizing air | 25 psi |
| Air volume | 60 cfm. |

The invention claimed is:

1. A solid composition comprising, by weight based on the total weight of the composition:
(a) from 5 to 45% of nisoldipine;
(b) from 50 to 90% of polyethylene oxide; said polyethylene oxide being a mixture comprising at least two polyethylene oxides with different viscosity range, at least one of a low viscosity in a range between 400 and 2,000 cp, and at least another of a high viscosity in a range between 4,000 and 20,000 cp; and
(c) the balance consisting of conventional additives.

2. The composition according to claim 1, wherein the low viscosity is in a range between 600 and 1200 cp, and the high viscosity is in a range between 8,800 and 17,600 cp.

3. The composition according to claim 1, wherein the nisoldipine is in a micronized form.

4. The composition according to claim 1, wherein the weight ratio low viscosity polyethylene oxide to high viscosity polyethylene oxide varies from 9:1 to 1:9.

5. The composition according to claim 1, wherein the weight ratio low viscosity polyethylene oxide to high viscosity polyethylene oxide is about 1:4.

6. The composition according to claim 1, wherein the parameter P of the polyethylene oxide blend which is defined as follows:
P=a.Log(x)+b.Log(y)
with:
a: amount of high viscosity polyethylene oxide in mg;
b: amount of low viscosity polyethylene oxide in mg;
x: viscosity of high viscosity polyethylene oxide in cp;
y: viscosity of low viscosity polyethylene oxide in cp;
for a+b equal to 200;
is comprised from 700 to 800.

7. The composition according to claim 6, wherein the parameter P is comprised from 750 to 780.

8. The composition according to claim 1 comprising, by weight, 5 to 25% of nisoldipine and 60 to 75% of PEO.

9. A solid composition comprising, by weight based on the total weight of the composition:
(a) from 5 to 25% of nisoldipine;
(b) from 60 to 75% of polyethylene oxide; said polyethylene oxide being a mixture comprising at least two polyethylene oxides with different viscosity range, at least one of a low viscosity in a range between 600 and 1,200 cp, and at least another of a high viscosity in a range between 8,800 and 17,600 cp; and
(c) the balance consisting of conventional additives;
wherein the weight ratio low viscosity polyethylene oxide to high viscosity polyethylene oxide is about 1:4.

10. The composition according to claim 9, wherein the nisoldipine is in a micronized form.

11. The composition according to claim 9, wherein the parameter P of the polyethylene oxide blend which is defined as follows:
P=a.Log(x)+b.Log(y)
with:
a: amount of high viscosity polyethylene oxide in mg;
b: amount of low viscosity polyethylene oxide in mg;
x: viscosity of high viscosity polyethylene oxide in cp;
y: viscosity of low viscosity polyethylene oxide in cp;
for a+b equal to 200;
is comprised from 700 to 800.

12. The composition according to claim 11, wherein the parameter P is comprised from 750 to 780.

13. The composition according to claim 1, in the form of tablets comprised of compressed granules.

14. The composition according to claim 9, in the form of tablets comprised of compressed granules.

15. A method for preparing the composition according to claim 1, comprising the steps of:
(i) mixing in the dry state and for a sufficient time, the active ingredient, the mixture of the two polyethylene oxides and optionally, one or several additives;
(ii) adding a solution with optionally one or several additives, followed by mixing for a sufficient period of time;
(iii) granulating the mixture obtained from step (ii); and
(iv) drying the granules thus formed for a sufficient period of time.

16. A method for preparing the composition according to claim 9, comprising the steps of:
(i) mixing in the dry state and for a sufficient time, the active ingredient, the mixture of the two polyethylene oxides and optionally, one or several additives;
(ii) adding a solution with optionally one or several additives, followed by mixing for a sufficient period of time;
(iii) granulating the mixture obtained from step (ii); and
(iv) drying the granules thus formed for a sufficient period of time.
